(19) United States
(12) Patent Application Publication
(10) Pub. No.: US 2023/0191059 A1
(43) Pub. Date: Jun. 22, 2023

(54) ENDOTRACHEAL TUBE CUFF WITH INTEGRATED SENSORS

(71) Applicants: BioVentures, LLC, Little Rock, AR (US); Arkansas Children's Hospital Research Institute, Little Rock, AR (US)

(72) Inventors: THOMAS NIENABER, Little Rock, AR (US); ADRIA ABELLA VILLAFRANCA, Little Rock, AR (US); KEVIN SEXTON, Little Rock, AR (US); JOSEPH SANFORD, Little Rock, AR (US); SARAH PEREZ, Little Rock, AR (US)

(21) Appl. No.: 17/923,511

(22) PCT Filed: May 4, 2021
(86) PCT No.: PCT/US2021/030678

§ 371 Date: Nov. 4, 2022

Related U.S. Application Data
(60) Provisional application No. 63/020,307, filed on May 5, 2020.

(51) Int. Cl. A61M 16/04

(52) U.S. Cl. CPC A61M 16/0438; A61M 2205/0294; A61M 2205/332

(57) ABSTRACT

Disclosed herein is an endotracheal tube cuff having a first layer, a second layer, and one or more sensors in a space between the first and second layers. The one or more sensors are operable to measure pressure between the endotracheal tube cuff and a tracheal wall of a patient. Also disclosed herein are methods of detecting a leak and preventing ischemia using the endotracheal tube cuff.

### CROSS-REFERENCE TO RELATED APPLICATIONS

This application claims priority to U.S. Provisional Application No. 63/020,307, filed May 5, 2020, the contents of which are entirely incorporated by reference herein.

### FIELD

The present disclosure relates to an endotracheal tube cuff with integrated sensors and methods of use thereof.

### BACKGROUND

Historically, uncuffed endotracheal tubes have been used in pediatric patients due to concern for complications surrounding the endotracheal tube (ETT) cuff. To date, there have been no significant advances in endotracheal tube design for neonates or infants that has been shown to decrease complications, especially problems due to prolonged intubations. It is currently thought that high pressure on the trachea—caused by the cuff—causes most of the complications. Pressure leads to venous blockade, leading to edema and/or necrosis which result in extubation failure, subglottic stenosis, bronchopulmonary dysplasia, etc. Supporting data for this mechanism is weak in adults and almost non-existent in children. There is only one pediatric-specific endotracheal tube, but this has not been shown to decrease complications significantly, likely due to the fact that the cuff pressure used is still greater than the neonatal central venous pressure.

Therefore, there is a need for an endotracheal tube cuff with integrated sensors to prevent the cuff pressure from impeding tracheal blood flow and decrease complications from intubation, particularly for the pediatric population.

### SUMMARY

This disclosure provides an endotracheal tube cuff with integrated sensors and methods of use thereof. The endotracheal tube cuff may include a first layer, a second layer, and one or more sensors in a space between the first and second layers. The one or more sensors are operable to measure pressure between the endotracheal tube cuff and a tracheal wall of a patient. In an aspect, the patient is a neonate.

In some aspects, the one or more sensors may be a piezoelectric sensor, a force sensitive resistor, or a force sensitive capacitor. The piezoelectric sensor may include a force sensitive resistor polymer. The space between the first and second layers may be filled with air or a saline solution. The one or more sensors may not be fixed to the first or second layer.

Also disclosed herein is a method of detecting a leak in a patient's ventilation system. The method may include placing an endotracheal tube cuff inside the patient's trachea, wherein the endotracheal tube cuff comprises a first layer, a second layer, and one or more sensors in a space between the first and second layers, inflating the endotracheal tube cuff, detecting, via the one or more sensors, if the cuff is too loose such that there is a leak of air, and adjusting the inflation of the endotracheal tube cuff if a leak is detected. In an aspect, the patient is a neonate.

In some aspects, the one or more sensors may be a piezoelectric sensor, a force sensitive resistor, or a force sensitive capacitor. The piezoelectric sensor may include a force sensitive resistor polymer. The space between the first and second layers may be filled with air, a saline solution, or any suitable fluid. The one or more sensors may not be fixed to the first or second layer.

Also described herein is a method of preventing ischemia in a patient. The method may include placing an endotracheal tube cuff inside the patient's trachea, wherein the endotracheal tube cuff comprises a first layer, a second layer, and one or more sensors in a space between the first and second layers, inflating the endotracheal tube cuff, detecting, via the one or more sensors, a pressure that the endotracheal tube cuff is exerting on the trachea wall, and adjusting the inflation of the endotracheal tube cuff based on the detected pressure. In an aspect, the patient is a neonate.

In an aspect, the method may further include measuring and/or calculating one or more additional physiologic parameters selected from blood flow, blood pressure, cardiac output, and/or heart rate. The one or more sensors may be a piezoelectric sensor, a force sensitive resistor, or a force sensitive capacitor. The piezoelectric sensor may include a force sensitive resistor polymer. The space between the first and second layers may be filled with air or a saline solution. The one or more sensors may not be fixed to the first or second layer.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIG. 1A is an example endotracheal tube with a cuff;

FIG. 1B is an example endotracheal tube with a cuff;

FIG. 2 is an example endotracheal tube cuff with an integrated sensor;

FIG. 3 is a graph of measured pressures from an example endotracheal tube cuff with an integrated sensor;

FIG. 4 is a summary of the data from an example endotracheal tube cuff with an integrated sensor that demonstrates the ability to detect when a leak is present, based on the analog readings from the sensor;

FIG. 5 is a graph of leak detection and respiratory rate for a rabbit with an ETT cuff in an example;

FIG. 6 shows a frequency analysis (Fast Fourier Transform) of a signal from a pressure sensor in an ETT cuff in an example;

FIG. 7A shows histology of a rabbit trachea from a control rabbit; and

FIG. 7B shows histology of a rabbit trachea from a rabbit with intervention from an ETT cuff.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Provided herein are endotracheal tube cuffs with integrated sensors and methods of use thereof to improve the safety and respiratory function of a patient. In some examples, the patient may be a pediatric patient, such as neonates. A cuffed ETT may have a lower incidence of ETT leak, improved ventilation, a decreased number of intubations, and/or use a smaller ETT through the cricoid. In some examples, the ETT cuff with integrated sensors may be operable to provide real-time pressure sensing and detect leaks, venous flow, respiratory rate, cardiac output, heart rate, and/or other physiologic parameters. The ETT cuff with integrated sensors may be an improvement over standard endotracheal cuffs because it may be used on neonatal patients, preventing pulmonary infections. It may also be used to prevent pulmonary infections and ischemia in adults or neonates, together with an accurate measurement/calculation of different physiologic parameters.

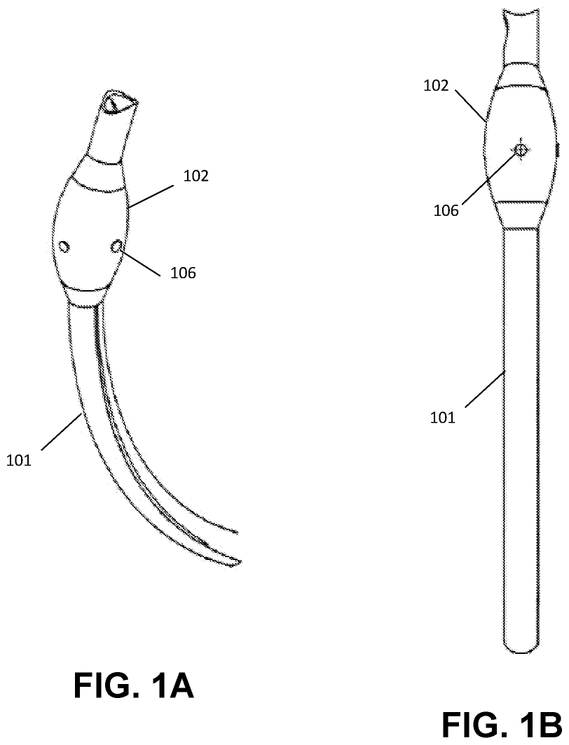

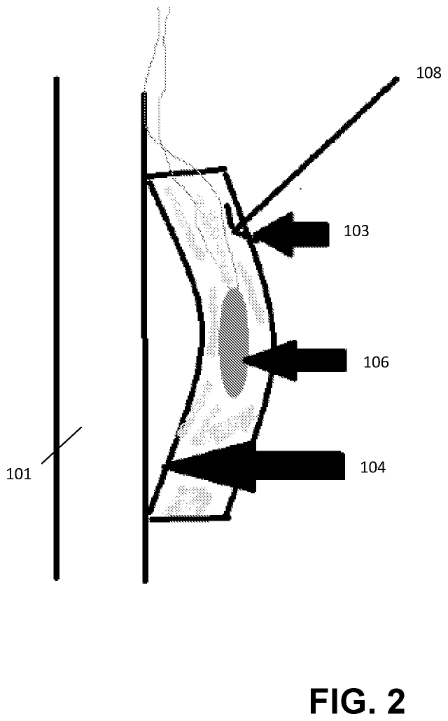

FIGS. 1A and 1B show an endotracheal tube 101 with a cuff 102. The ETT cuff 102 may include one or more sensors 106 integrated into the ETT cuff. In some examples, the ETT cuff may include one or more layers. For example, the ETT cuff may include 1, 2, 3, or 4 layers. The one or more sensors may be integrated with or proximal to the one or more layers. In some examples, the one or more sensors may be in a space between two or more layers. The one or more sensors may not be fixed to the two or more layers. In at least one example, the ETT cuff may be a double layer cuff. In an example, the ETT cuff may be a high volume, low pressure (HVLP) cuff designed to spread the pressure over a large area. The multi-layered cuff may be filled with air or other liquid such as saline solution, with the one or more sensors between the layers, as seen in FIG. 2. FIG. 2 is an example double layer cuff 102 with a first layer 103, a second layer 104, and a sensor 106 between the first layer 103 and the second layer 104. The space within the first layer 103 and the second layer 104 is also filled with air or liquid 108.

The ETT cuff, or one or more layers of the ETT cuff, may be made of a biocompatible polymer. In some examples, the ETT cuff material may be an ultrathin, high tensile strength material. Non-limiting examples of materials the ETT cuff may be made of include micro-thin polyvinyl chloride (PVC) and/or ultrathin polyurethane. The layers may be made of the same or different materials.

The sensors may be thin enough so that they fit in the cuff structure and may be inexpensive to manufacture. In some examples, the thickness of the sensors may be less than 0.2 mm thick. The sensors may be placed at various positions along the cuff to cover different angles of the cuff. In some examples, the one or more sensors may be on or embedded within a single layer of the cuff, such as sensors being integrated in the cuff material itself. In other examples, the one or more sensors may sit between two layers of the cuff such that they are not fixed to any layer or spot in the cuff, as seen in FIG. 2. The one or more sensors may freely bend inside the area between the cuff layers. In various examples, the ETT cuff may include at least 1, at least 2, at least 3, at least 4, or at least 5 sensors. The sensors may be wired or wireless.

The one or more sensors may be force-sensing resistance sensors, flow sensors, carbon dioxide (CO₂) sensors, and/or ultrasound sensors. Non-limiting examples of force sensors include a piezoelectric sensor, a force sensitive resistor, a strain gauge sensor, a force sensitive capacitor, or any pressure sensor capable of measuring force. In an example, the one or more sensors may be electrically conductive, such that they are operable to react to pressure/force applied to it, such as piezoelectric sensor. In some examples, the one or more integrated sensors may be a force sensitive resistor polymer. A force sensitive resistor polymer may have a lower manufacturing cost than using more complex piezoelectric sensors. In at least one example, the one or more integrated sensors may include a polymeric foil (polyolefins) impregnated with carbon black.

The one or more sensors are located inside the cuff such that they are operable to measure the pressure that the cuff is exerting on the trachea walls. The one or more sensors may provide real-time pressure sensing. In some examples, the integrated sensors may be operable to detect changes in pressure of the cuff and changes in blood flow in the tracheal wall. For example, the integrated sensors in the ETT cuff may be operable to detect and control pressure in the ETT by detecting changes in compliance. The integrated sensors may further be used to maintain cuff pressures below the limit of occluding venous flow, which may minimize the risk of subglottic stenosis.

In additional examples, the one or more sensors may further be operable to measure and/or calculate additional parameters including but not limited to venous flow, heart rate, respiratory rate, blood pressure, cardiac output, and/or other physiologic parameters. In at least one example, the one or more sensors in the ETT cuff may be operable to detect venous blood flow in the tracheal mucosa. The one or more sensors in the ETT cuff may further be operable to detect pressures in nearby structures or other blood vessels (e.g. large changes in pressure in the aorta may indicate a PDA).

The ETT cuff may be circular, i.e. symmetrical, cylindrical, oval, or any shape that can adapt to the trachea shape. However, this may not be the best shape, as the trachea is not a circular shape. In some examples, the endotracheal cuff may have a dampening cuff pressure design. In some examples, the cuff shape may be as important as cuff pressure for creating a seal in the trachea.

In some examples, the ETT cuff may include one or more separate compartments. The compartments may be located inside the cuff and may be inflated or de-inflated depending on the trachea. For example, if air is leaking along the posterior aspect of the ETT cuff, only the posterior part of the cuff can be inflated using the compartment. In some examples, the compartments may contain a senor or a set of sensors. The cuff compartments or sections may be used to detect which region of the cuff requires additional inflation. Adding compartments/sections may increase the profile of cuff, but utilizing an ultrathin, high tensile strength material may decrease the profile, ideally making the cuff flush with the ETT when deflated.

Data from the sensors (e.g. one or more of the measured physiologic parameters) may be collected and transmitted through a wired connection or a wireless connection to a data collection system. In some examples, the data collection system may be a computer or a medical machine (e.g. a ventilator). The data may be being processed though a set of algorithms stored on the data collection system to detect if there is an air leak on the ventilator. The data may also be used to calculate heart rate and/or cardiac output. To calculate the heart rate, the data signal is transferred to the frequency domain using Fast Fourier Transform to detect the harmonics of the signal. The heart rate frequency is reflected on the frequency harmonics. Based on the frequency, the heart rate may be calculated. At least 10 seconds may be needed to calculate the actual heart rate based on the signal. The larger the signal being processed on the frequency domain, the better accuracy on calculating heart rate.

The data collection system may use machine learning and/or artificial intelligence algorithms (e.g. a convolutional neural network with an aggregated Long Short Term Memory algorithm) to classify signals (leak vs no leak) and/or to calculate heart rate and/or cardiac output.

The data may be displayed in real-time on a display in communication with the data collection system for a healthcare professional. The communication between the display and the data collection system may be wired or wireless. In some examples, the display may be part of the data collection system (e.g. a computer). In other examples, the display may be part of a separate electronic device.

Provided herein are methods of detecting a leak in a patient's ventilation system when the ETT is within a patient's trachea. The sensors may detect when there is a leak inside the ventilation system depending on the force that the cuff is exerting on the trachea wall. The data from the sensors may then be transmitted to a data collection system where data from the sensors may be analyzed or used to calculate and/or display one or more physiological parameters. When the ETT is placed inside a patient's trachea, the physician then inflates the cuff and auscultates to detect if there is a leak of air (i.e. air that is coming from the ventilator to the lungs of the patient). If the cuff is too loose, there will be a leak and air will escape from the lungs of the patient to the exterior instead of coming all back to the ventilator machine. The present ETT cuff is operable to detect this leak of air, since the one or more sensors are operable to detect this air flowing out between the trachea of the patient and the cuff.

Provided herein are methods of preventing ischemia by measuring pressure that an endotracheal tube cuff is exerting on the trachea wall. The pressure is measured using the one or more sensors integrated within or between one or more layers of the ETT cuff. The inflation of the ETT cuff may then be adjusted based on the measured pressure to prevent ischemia. The methods may further include measuring and/or calculating one or more additional physiologic parameters, including but not limited to blood flow, blood pressure, cardiac output, and/or heart rate. The ETT cuff with integrated sensors may then be further used to prevent ischemia on the trachea walls by monitoring these parameters.

### EXAMPLES

### Example 1

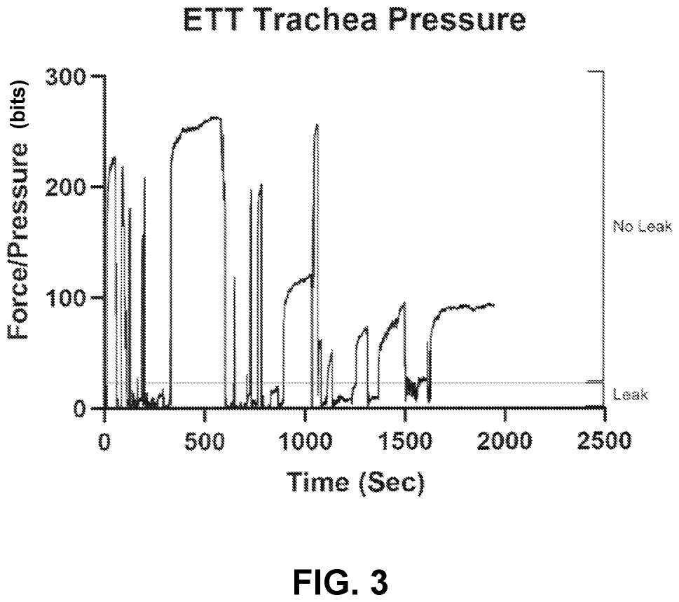

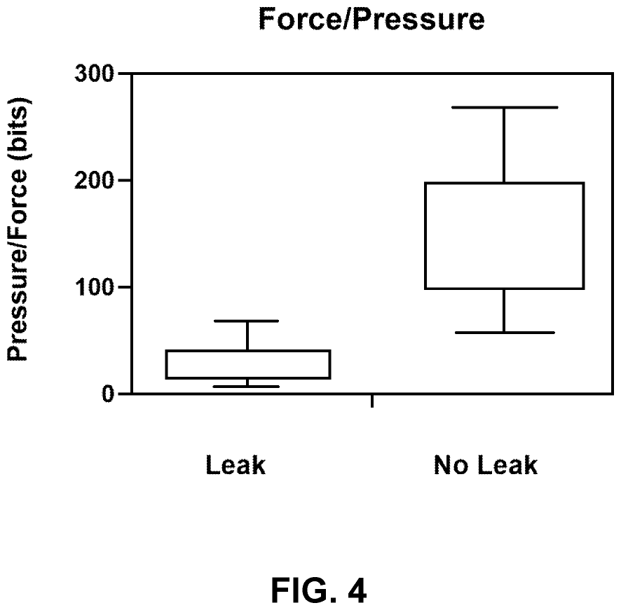

A prototype ETT cuff with a sensor was tested in a mannequin model (adult) with a mechanical ventilator. FIG. 3 is a graph of measured pressures from the prototype. FIG. 4 is a summary of the data from the prototype that demonstrates the ability to detect when a leak is present, based on the analog readings from the sensor. The results show a relationship between the electrical analog reading represented in bits, and the binary answer of detected leak (leak or no leak). The binary answer to detect a leak, was made through audible detection of the leak.

Results of the in vitro testing have demonstrated the surprising findings of a consistent cuff leak at a certain cuff volume regardless of the pressure used to drive ventilation. These data suggest that cuff shape and cuff pressure may be important for creating a seal in the trachea and a dampening cuff pressure design may be better compared to a traditional cuff design.

### Example 2

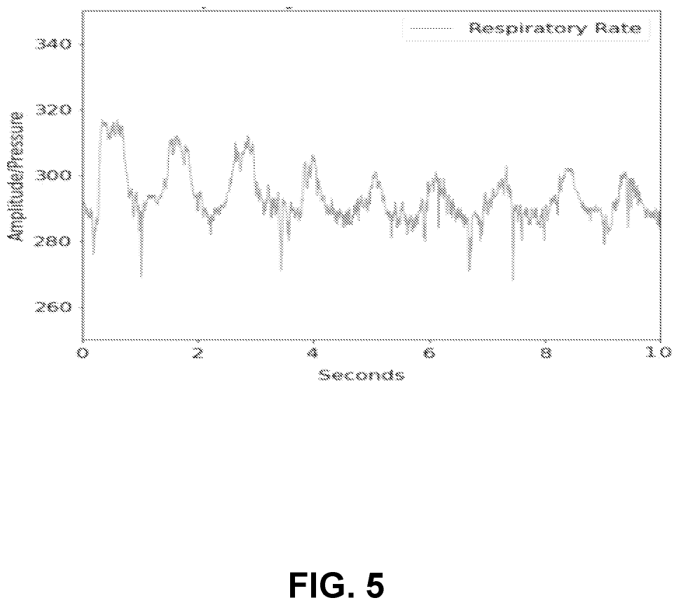

The prototype ETT cuff was further tested on 16 rabbits, 8 in each group (control and intervention). In a spontaneously breathing rabbit with a leak, the respiratory rate was seen, which has also been seen in mannequin and animals when a leak is present. FIG. 5 shows leak detection and respiratory rate for an exemplary rabbit. This rabbit was breathing at 50-60/min. Here the sensor provided a signal with a familiar pattern and a rate of approximately 55/min.

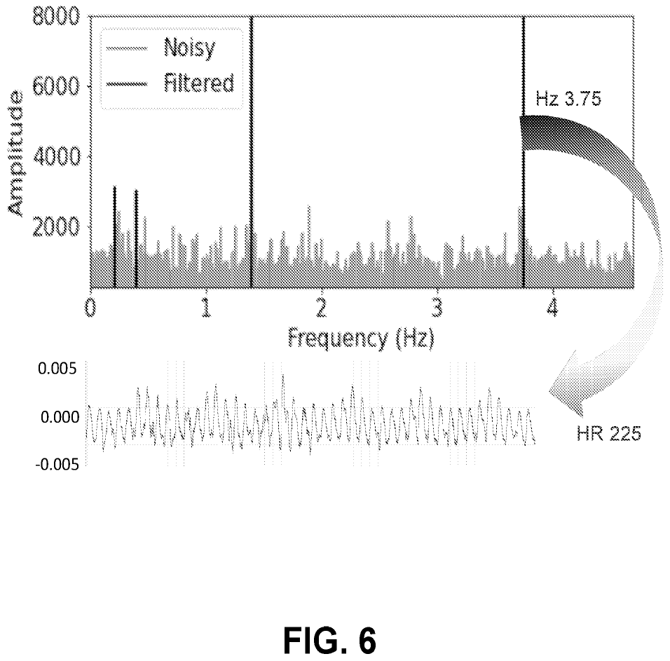

FIG. 6 shows frequency analysis (Fast Fourier Transform) of the signal from the sensor. Several consistent signals were observed, especially at the point of no leak detection. The strongest signal was at a frequency of approximately 3.75 Hz, which corresponds to the EKG tracing at the same time, at a heart rate of 225 bpm.

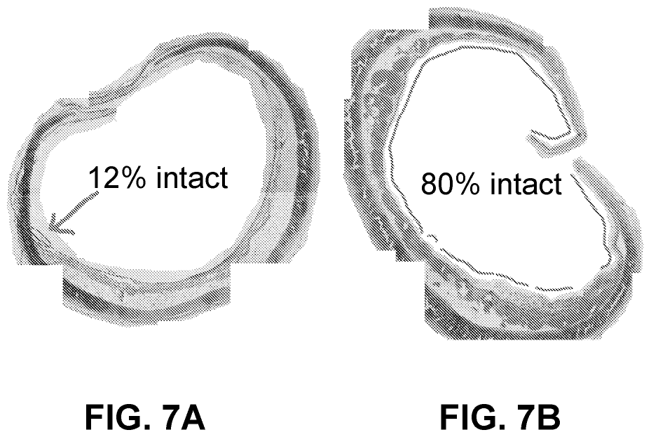

FIGS. 7A and 7B show histology of rabbit trachea from a control rabbit (FIG. 7A) and a rabbit with intervention (FIG. 7B). In the control, it can be seen that the epithelium and cilia about 12% intact. In the rabbit with intervention with the ETT cuff, it can be seen that the epithelium and cilia are about 80% intact and after 2 hours of intubation.

Having described several embodiments, it will be recognized by those skilled in the art that various modifications, alternative constructions, and equivalents may be used without departing from the spirit of the disclosure. Additionally, a number of well-known processes and elements have not been described in order to avoid unnecessarily obscuring the present disclosure. Accordingly, the above description should not be taken as limiting the scope of the disclosure.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. An endotracheal tube cuff comprising:
a first layer; and
one or more sensors integrated with or proximal to the first layer, wherein the one or more sensors are operable to measure pressure between the endotracheal tube cuff and a tracheal wall of a patient.

2. The endotracheal tube cuff of claim 1, further comprising:
a second layer surrounding the first layer,
wherein the one or more sensors are in a space between the first and second layers.

3. The endotracheal tube cuff of claim 2, wherein the space between the first and second layers is filled with air or a saline solution.

4. The endotracheal tube cuff of claim 2, wherein the one or more sensors are not fixed to the first or second layer.

5. The endotracheal tube cuff of claim 1, wherein the one or more sensors is a piezoelectric sensor, a force sensitive resistor, or a force sensitive capacitor.

6. The endotracheal tube cuff of claim 5, wherein the piezoelectric sensor comprises a force sensitive resistor polymer.

7. The endotracheal tube cuff of claim 1, wherein the patient is a neonate.

8. A method of detecting a leak in a patient's ventilation system, the method comprising:
placing an endotracheal tube cuff inside the patient's trachea, wherein the endotracheal tube cuff comprises a first layer, a second layer, and one or more sensors in a space between the first and second layers;
inflating the endotracheal tube cuff;
detecting, via the one or more sensors, if the cuff is too loose such that there is a leak of air; and
adjusting the inflation of the endotracheal tube cuff if a leak is detected.

9. The method of claim 8, wherein the one or more sensors is a piezoelectric sensor, a force sensitive resistor, a force sensitive capacitor, or a strain gauge sensor.

10. The method of claim 9, wherein the piezoelectric sensor comprises a force sensitive resistor polymer.

11. The method of claim 8, wherein the space between the first and second layers is filled with air or a saline solution.

12. The method of claim 8, wherein the one or more sensors are not fixed to the first or second layer.

13. The method of claim 8, wherein the patient is a neonate.

14. A method of preventing ischemia in a patient, the method comprising:
placing an endotracheal tube cuff inside the patient's trachea, wherein the endotracheal tube cuff comprises a first layer, a second layer, and one or more sensors in a space between the first and second layers;
inflating the endotracheal tube cuff;
detecting, via the one or more sensors, a pressure that the endotracheal tube cuff is exerting on the trachea wall; and
adjusting the inflation of the endotracheal tube cuff based on the detected pressure.

15. The method of claim 14 further comprising measuring and/or calculating one or more additional physiologic parameters selected from blood flow, blood pressure, cardiac output, and/or heart rate.

16. The method of claim 15, wherein the one or more sensors is a piezoelectric sensor, a force sensitive resistor, a force sensitive capacitor, or a strain gauge sensor.

17. The method of claim 16, wherein the piezoelectric sensor comprises a force sensitive resistor polymer.

18. The method of claim 14, wherein the space between the first and second layers is filled with air or a saline solution.

19. The method of claim 14, wherein the one or more sensors are not fixed to the first or second layer.

20. The method of claim 14, wherein the patient is a neonate.
